(19) United States
(12) Patent Application Publication
(10) Pub. No.: US 2007/0286810 A1
(43) Pub. Date: Dec. 13, 2007

(54) Nanoparticles comprising lanthanide chelates

(75) Inventors: Jari Hovinen, Raisio (FI); Veli-Matti Mukkala, Kaarina (FI); Harri Hakala, Turku (FI)

Correspondence Address: BUCHANAN, INGERSOLL & ROONEY PC, POST OFFICE BOX 1404, ALEXANDRIA, VA, 22313-1404, US

(73) Assignee: WALLAC OY, Turku (FI)

(21) Appl. No.: 11/448,778

(22) Filed: Jun. 8, 2006

(51) Int. Cl. A61K 49/10; C07F 5/00; C08F 277/00

(52) U.S. Cl. 424/9.322; 534/16; 525/245

(57) ABSTRACT

The invention concerns a method for preparing a particle comprising a lanthanide chelate, comprising polymerizing a lanthanide chelate derivative with one or more monomers, wherein the lanthanide chelate derivative has the formula M-L-Y (I), wherein Y is a lanthanide chelate; L is a linker and M is a polymerizable moiety.

The invention concerns also the novel lanthanide chelate derivative M-L-Y (I). Further, the invention concerns a novel particle comprising a lanthanide chelate covalently bound to an organic polymer or copolymer via a linker.

### FIELD OF THE INVENTION

This invention relates to particles comprising luminescent lanthanide chelates and to a method for their preparation.

### BACKGROUND OF THE INVENTION

The high specific activity and very low background signal has made time-resolved fluorescence (TR-F) based on lanthanide(III) chelates a succesful detection technology for a variety of analytes. Indeed, lanthanide(III) chelates have been used in in vitro diagnostics over two decades.

The commercially available DELFIA® assay method uses a non-luminescent europium(III) chelate for detecting a target molecule. The usual steps of a DELFIA® assay include capturing of the target molecule to a surface using a target-specific antibody associated with a lanthanide chelate, such as a europium (III) chelate, washing away of contaminating materials, and signal enhancement. For the signal enhancement, europium(III) ion is dissociated from the non-luminescent chelate by lowering pH to 3.2 and the luminescence is enhanced with a mixture of β-diketone (4,4,4-trifluoro-1-(2-napthyl)-butane-1,3-dione), detergent (Triton X) and chelator (trioctyl phosphine oxide, TOPO). The new chelate formed has a very high luminescence, giving detection sensitivity ca 50·10⁻¹⁵ M.

Luminescent lanthanide(III) chelates with increased stability have been developed more recently. These chelates consist of a ligand with a reactive group for covalent conjugation to bioactive molecules, an aromatic structure, which absorbs the excitation energy and transfers it to the lanthanide ion and additional chelating groups such as carboxylic acid moieties and amines.

Further improvement to lanthanide(III) chelate materials and methods would allow for development of more sensitive assay methods, and increased ability to detect target molecules.

### SUMMARY OF THE INVENTION

In one aspect, the invention provides a method preparing a particle comprising a lanthanide chelate, wherein a lanthanide chelate derivative of the formula M-L-Y (I), wherein Y is a lanthanide chelate; L is a linker and M is a polymerizable moiety, is allowed to polymerize with one or more monomers.

According to another aspect, the invention provides a lanthanide chelate derivative of the formula M-L-Y (I), wherein Y is a lanthanide chelate; L is a linker and M is a polymerizable moiety.

According to a third aspect, the invention provides a particle based on a compound of formula Z-L-Y (II)

wherein,

Z is an organic polymer or copolymer

L is a linker; and

Y is a lanthanide chelate,

wherein the lanthanide chelate Y is covalently bound to Z via the linker L.

### DETAILED DESCRIPTION OF THE INVENTION

The technology described herein relates to particles linked covalently to luminescent lanthanide(III) chelates. The particles are prepared by polymerizing lanthanide(III) chelate derivatives in the presence of monomers. Because the resultant particles are covalently bound to the lanthanide(III) chelates, the signal obtained generally does not decrease as the function of time due to leaking upon storage. Because the lanthanide(III) chelates are luminescent, no additives that promote luminescence, such as phosphine oxides, are needed in the polymerization matrix. In addition, because the beads can be made using organic polymers or copolymers, such as polystyrene, they can be stable under basic conditions.

It has been shown previously that lanthanide (III) chelate detection sensitivity can be enhanced by incorporating chelates into particles. Beads containing lanthanide(III) chelates have most commonly been prepared simply by swelling the chelates into the polymer, as is described, for example, in Cummins, C. M., Koivunen, M. E., Stephanian, A., Gee, S. J., Hammock, B. D., Kennedy, I. M., 2006, Biocencors and Bioelectronics, 21, 1077. In addition, silica based nanobeads have been described by Hai, X., Tan, M., Wang, G., Ye, Z., Yuan, J., Matsumoto, K., 2004, Anal. Sci., 20, 245 and Sun, X., Wuest, M., Kovacs, Z., Sherry, A. D., Motekaitis, R., Wang, Z., Martell, A. E., Welch, M. J., Anderson, C. J., 2003, J. Biol. Inorg. Chem., 8, 217. Particles produced using nanodispersions of particle sizes in the middle or lower nanometer range (50-500 nm) are described, for example, in Horn, D., Rieger, J., 2001, Angew. Chem. Int. Ed. Engl., 40, 4330.

The invention provides a method for preparing particles comprising a lanthanide chelate. The method involves polymerizing a lanthanide chelate derivative with one or more monomers, wherein the lanthanide chelate derivative has the formula M-L-Y (I), wherein Y is a lanthanide chelate; L is a linker and M is a polymerizable moiety.

In an embodiment, the polymerizable moiety M in the lanthanide chelate derivative of formula (I) is a pendent vinyl group, acrylate group, methacrylate group, ethacrylate group, 2-phenylacrylate group, vinylketone group, acrylamide group, methacrylamide group, itaconate group or a styrene group. Specific examples include methacrylamide or acrylamide.

The compound of formula (I) can be allowed to polymerize with one or more organic monomers. Examples of organic monomers include styrene, vinyl alcohol, acrylic acid, metacrylic acid and esters and amides derived thereof. A specific example monomer is styrene.

The lanthanide chelate can be a luminescent lanthanide chelate. In an embodiment, the lanthanide chelate derivative is typically made of a chromophoric moiety comprising one or more aromatic units, and a chelating part. Exemplary compounds include those of shown below:

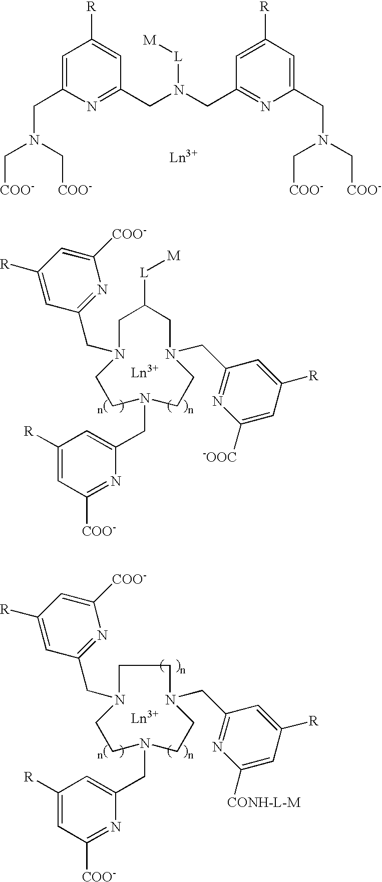

wherein

R is independently furyl, thiophenyl, or trialkoxyphenyl;

n is independently 1 or 2; and

L is a linker; and

Ln is europium, terbium, samarium, dysprosium or gadolinium; and

M is a polymerizable moiety.

The linker -L- is formed from one to ten moieties, each moiety being selected from the group consisting of phenylene, alkyl containing 1-12 carbon atoms, ethynediyl(—C≡C—), ethylenediyl(—C═C—); ether (—O—), thioether (—S—), amide (—CO—NH— and —NH—CO— and —CO—NR′ and —NR′—CO—), carbonyl(—CO—), ester (—COO— and —OOC—), disulfide (—SS—), diaza, (—N═N—), thiourea (—NH—CS—NH—) or a tertiary amine (—NR′—), where R′ represents an alkyl containing less than 5 carbon atoms.

The polymerization can be performed in the presence of a cross-linking agent such as divinylbenzene.

The size of the particle is generally below 500 nm, such as below 300 nm. In an embodiment, the diameter of the particle is in the range 10 . . . 150 nm, such as 90 . . . 120 nm.

The organic polymer or copolymer of the particle can be constructed, for example, of from monomers of vinyl, acrylate, methacrylate, ethacrylate, 2-phenylacrylate, vinylketone, vinyl alcohol, acrylamide, methacrylamide, itaconate or styrene.

### EXAMPLES

The technology described herein is further elucidated by the following non-restricting examples. The structures and synthetic routes employed in the experimental part are depicted in Schemes 1 and 2. Experimental details are given in Examples 1-9. Scheme 3 describes a schematic preparation of a polymerizable terbium chelate. Properties of the nanospheres prepared are collected in Table 1.

### Procedures

Adsorption column chromatography was performed on columns packed with silica gel 60 (Merck). All dry solvents were from Merck and they were used as received. NMR spectra were recorded on a Brucker 250 on a Jeol LA 400 spectrometer operating at 250.13 and 399.7 MHz for ¹H, respectively. The signal of TMS was used as an internal reference. Coupling constants are given in Hertz. ESI-TOF mass spectra were recorded on an Applied Biosystems Mariner instrument. Luminescence measurements were measured with a PerkinElmer LS-5 luminescence spectrometer. IR and UV-spectra spectra were recorded on a PerkinElmer Spectrum One and Shimatzu 2400 instruments, respectively. Particle size analyses were performed on a Coulter LS-230 instrument, and are based on volume statistics. Photophysical properties of the nanoparticles prepared were measured as disclosed in Latva, M., Takalo, H., Mukkala, V.-M., Matachescu, C., Rodriques-Ubis, J. C., Kankare, J., 1997, J. Lumin., 35, 149 but the chelate concentration measurements were based on the weight of dry beads.

### Example 1

### The Synthesis of N-(3-acetylphenyl)methacrylamide (1)

3-Aminoacetophenone (20.9 g, 0.15 mol) was dissolved in dry pyridine (60 mL) on an ice-water bath. Methacryloyl chloride (26.1 mL, 0.24 mol) was added dropwise during ½ h, and the mixture was stirred for an additional ½ h. The stirring was continued for 3 h at RT. All volatiles were removed in vacuo. The residue was dissolved in dichloromethane (150 mL), washed with 0.5 M HCl (2·100 mL) and water (2·100 mL) and dried (Na₂SO₄). Purification was performed on silica gel. The column was first eluted with CH₂Cl₂ to elute fast migrating impurities, and then with 5% (v/v) methanolic dichloromethane to elute the product. Yield was 18.1 g (59%). ¹H NMR (CDCl₃): 8.10 (1H, m); 8.00 (1H, br); 7.95 (1H, m); 7.43 (1H, m); 7.23 (1H, m); 5.85 (1H, s); 5.51 (1H, s); 2.61 (3H, s); 2.08 (3H, s). ESI-TOF MS: required for C₁₂H₁₄NO₂⁺ 204.10 (M+H),⁺ found 204.08.

### Example 2

### The Synthesis of 4,4,4-Trifluoro-1-[3-(methacrylamido)phenyl]-1,3-butanedione (2)

To a strirred solution of compound 1 (11.9 g, 58.55 mmol) in dry THF (100 mL) was added portionwise sodium hydride (3.52 g, 88 mmol; 60% dispersion in oil). After 5 min, ethyl trifluoroacetate (13.9 mL, 0.18 mol) was added, and the mixture was strirred for an additional 1 h before being concentrated in vacuo. The residue was suspended in ethyl acetate (220 mL) and acidified with 10% aqueous H₂SO₄ (80 mL), and washed with water. The organic layer was separated and dried over Na₂SO₄. Purification on silica gel (eluent, petroleum ether, bp. 40-60° C./ethyl acetate, 1:1, v/v) yielded 8.71 g (50%) of the title compound. ¹H NMR (CDCl₃): 8.14 (1H, s); 7.87 (1H, d, J 7.5); 7.74 (1H, br s); 7.74 (1H, d, J 7.8); 7.48 (1H, t, J 8.0); 6.58 (1H, s); 5.85 (1H, s); 5.54 (1H, s); 2.09 (3H, s). λₘₐₓ(EtOH)/nm: 208, 250, 326. ESI-TOF MS: required for C₁₃H₁₃F₃NNaO₃⁺ 322.07 (M+Na),⁺ found 322.03.

### Example 3

### The Synthesis of the Europium Chelate (3)

Compound 2 (9.8 g, 32.7 mmol) was dissolved in the mixture of abs. ethanol (100 mL) and piperidine (3.2 mL), and the mixture was warmed to 45° C. Europium chloride hexahydrate (2.40 g, 6.54 mmol, predissolved in 20 mL of water) was added dropwise. The mixture was allowed to cool to RT, and water (100 mL) was added dropwise. The precipitation formed was collected by filtration, washed with water, and dried in vacuo. Yield was 7.9 g. λₘₐₓ(H₂O+1% DMF, v/v)/nm: 326 (ε 50552). ν/ₘₐₓ(KBr) cm⁻¹: 3440, 1663, 1620, 1586, 1534, 1489, 1301, 1187, 1138, 780, 580. Exₘₐₓ 614 nm; Emₘₐₓ 353 nm (tris-saline buffer, pH 7.75).

### Example 4

### The Synthesis of tetra-(tert-butyl)-2,2′,2″,2′″-{[6-(tert-butyloxycarbonylamino)hexylimino]bis(methylene)bis(4-bromopyridine-6,2-diyl)bis methylenenitrilo)}tetrakis(acetate) (5)

4-Bromo-6-bromomethyl-2-pyridylmethylenenitrilobis(acetic acid) di(tert-butyl ester) (4, 8.50 g, 16.7 mmol) and 6-tert-butoxycarbamoylhexane-1,6-diamine (1.80 g, 8.4 mmol) were dissolved in dry acetonitrile (60 mL). K₂CO₃ (9.2 g, 66.8 mmol) was added, and the mixture was heated overnight at 50° C. The precipitation formed was removed by filtration, and the filtrate was concentrated. Purification on silica gel (eluent: petroleum ether, bp 40-60° C.: ethyl acetate, from 10:1 to 5:2, v/v) yielded 5.9 g (65%) of compound 5. ¹H NMR (CDCl₃): δ 7.73 (2H, d, J 1.9); 7.58 (2H, d, J 1.9); 4.00 (4H, s); 3.74 (4H, s); 3.47 (8H, s); 3.08 (2H, q, J 5.5); 2.51 (2H, t, J 7.2); 1.46 (36H, s); 1.44 (9H, s); 1.53-1.42 (4H, m); 1.33-1.22 (4H, m). ν/ₘₐₓ(film) cm⁻¹: 3401 (N—H); 1734 (C═O); 1565 (arom. C—C). λₘₐₓ(EtOH)/nm 268. ESI-TOF MS for C₄₉H₇₈Br₂N₆O₁₀ (M+2H)²⁺: calcd, 536.22; found, 536.18.

### Example 5

### The Synthesis of tetra-(tert-butyl)-2,2′,2″,2′″-{[6-(tert-butyloxycarbonylamino)hexylimino]bis(methylene)bis(4-(thiophen-2-yl)pyridine-6,2-diyl)bis methylenenitrilo)}tetrakis(acetate) (6)

Compound 5 (2.85 g, 2.66 mmol) and 2-(tributylstannyl)-thiophene (1.86 mL, 5.86 mmol) were dissolved in dry DMF (25 mL) and deaerated with argon. (Ph₃P)₄Pd (0.215 g, 0.22 mmol) was added, and the mixture was stirred at 90° C. for 6 h in dark. The mixture was cooled to room temperature and concentrated in vacuo. Purification was performed on silica gel (eluent: petroleum ether, bp 40-60° C.: ethyl acetate: triethylamine, from 5:1:1 to 5:3:1, v/v/v). Yield was 2.2 g (76%). ¹H NMR (CDCl₃): δ 7.76 (2H, s); 7.70 (2H, s); 7.55 (2H, d J 3.1); 7.36 (2H, d, J 4.9); 7.09 (2H, m); 4.05 (4H, s); 3.82 (4H, s); 3.50 (8H, s); 3.03 (2H, m); 2.60 (2H, m); 1.49-1.43 (4H, m); 1.45 (36H, s); 1.42 (9H, s); 1.39-1.32 (4H, m). νₘₐₓ (film)/cm⁻¹ 1730 (C═O). λₘₐₓ(EtOH)/nm 293. ESI-TOF-MS for C₅₇H₈₅N₆O₁₀S₂(M+2H)²⁺: calcd, 539.29; obsd, 539.23.

### Example 6

### The Synthesis of 2,2′,2″,2′″-{(6-Aminohex-1-yl-imino)bis(methylene)bis[4-(thiophen-2-yl)pyridine-6,2-diyl)]bis(methylenenitrilo)}tetrakis(acetic acid) (7)

Compound 6 (2.16 g, 2.00 mmol) was dissolved in trifluoroacetic acid (25 mL), and the mixture was stirred for 2 h at room temperature before being concentrated. The residue was triturated with diethyl ether. The precipitation formed was filtered, washed with diethyl ether and dried in vacuo. Yield was quantitative. ¹H NMR (DMSO-d₆): 7.90 (2H, s); 7.78 (2H, d, J 4.9); 7.73 (2H, d, J 3.4); 7.71 (2H, s); 7.25 (2H, dd, J 3.4 and 4.9); 3.95 (4H, s); 3.30 (8H, s); 3.22 (2H, m); 2.74 (2H, m); 1.78 (2H, m); 1.50 (2H, m); 1.33-1.23 (4H, m); νₘₐₓ(KBr)/cm⁻¹ 1735, 1675, 1609 (C═O); 1559 (arom. C—C). λₘₐₓ(EtOH)/nm 300.

### Example 7

### The Synthesis of 2,2′,2″,2′″-{(6-aminohex-1-yl-imino)bis(methylene)bis[4-(thiophen-2-yl)pyridine-6,2-diyl)]bis(methylenenitrilo)}tetrakis(acetic acid) europium(III) (8)

Compound 7 (1.9 g) was dissolved in water (30 mL), and pH of the solution was adjusted to 6.5 with solid NaHCO₃. Europium chloride hexahydrate (0.81 g, 2.2 mmol; predissolved in 30 mL of water) was added dropwice keepimg pH at ca 6. The mixture was stirred for 1.5 h at RT. pH was rised to 8.5 with aq. NaOH. The precipitation was removed by centrifugation. The clear solution was collected and concentrated in vacuo. It was used for the next step without further purification. νₘₐₓ(KBr)/cm⁻¹ 1684, 1638, 1615 (C═O); 1552 (arom. C—C). λₘₐₓ(EtOH)/nm 312. ESI-TOF-MS for C₃₆H₄₀EuN₆O₈S₂⁻(M−H)⁻: calcd, 901.16; obsd, 901.16.

### Example 8

### The Synthesis of 2,2′,2″,2′″-{(6-methacroylamidohex-1-yl-imino)bis(methylene)bis[4-(thiophen-2-yl)pyridine-6,2-diyl)]bis(methylenenitrilo)}tetrakis(acetic acid) europium(III) (9)

Compound 8 (2.6 g, 2.8 mmol) was dissolved in the mixture of water (20 mL), THF (40 mL) and DIPEA (1.7 mL). Methacroyl chloride (0.42 g, 4.0 mmol) was added, and the mixture was stirred for 5 min at RT before being concentrated in vacuo. The residue was suspended in chloform (30 mL). The precipitation formed was removed by filtration. The filtrate was concentrated to give the title compound. ESI-TOF-MS for C₄₀H₄₄EuN₆O₉S₂⁻ (M−H)⁻: calcd, 969.18; obsd, 969.18. The partition coefficient of compound 9 between H₂O and CHCl₃, was ca 1:1.

### Example 9

### Polymerization. A Typical Procedure

A mixture of styrene (1.83 mL), acrylic acid (234 mm³), hexadecane (94 mg), divinyl benzene (0.12 g) and compound 3 (0.512 g, 0.358 mmol; 20% of the dry weight; predissolved in 2.0 mL of chloroform) and TOPO (0.208 g, 0.534 mmol) were dissolved in water (40.0 mL) containing sodium dodecyl sulfate (0.09 g) and sodiumborate decahydrate (0.017 g). The resulting suspension was deaerated with argon and homogenized using ultrasound (1 min, 215 W). The resulting emulsion was transferred into a reactor, and it was stirred mechanically (140 rpm) at 60° C. for 20 min under argon (pH 3). The polymerization was initiated by addition of potassium persulphate (0.05 g, predissolved in 3.00 mL of degassed water). The reaction was allowed to proceed for 5 h. The mixture was allowed to cool to RT and purified by dialysis. The following beads were prepared:

10% (w/w) chelate 3 [bead A]

20% (w/w) chelate 3 [bead B]

20% (w/w) chelate 3+1.5 equiv. TOPO, [bead C]

27% (w/w) chelate 9 [bead D]

where % w/w is the of the weight of chelate from the dry weight of the polymerization mixture.

TABLE 1
Properties of the nanospheres prepared.

| Bead | size (nm) | chelate (w/w)^{a} | relative fluorescence^{b} |
|---|---|---|---|
| A | 101 | 3 (10%) | 0.097^{c} (0.19)^{d} |
| B | 115 | 3 (20%) | 0.210 (0.39) |
| C | 111 | 3 (20%) + TOPO | 1.52 (1.40) |
| D | 93 | 9 (27%) | 0.42 (0.42) |

^{a}percentage of dry weight of the chelate in the polymerization mixture;
^{b}compared to DELFIA enhancement solution;
^{c}measured in Tris-saline buffer, pH 7.75;
^{d}Tris-saline buffer, pH 7.75 + TOPO + Triton-X.

It will be appreciated that the methods described herein can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

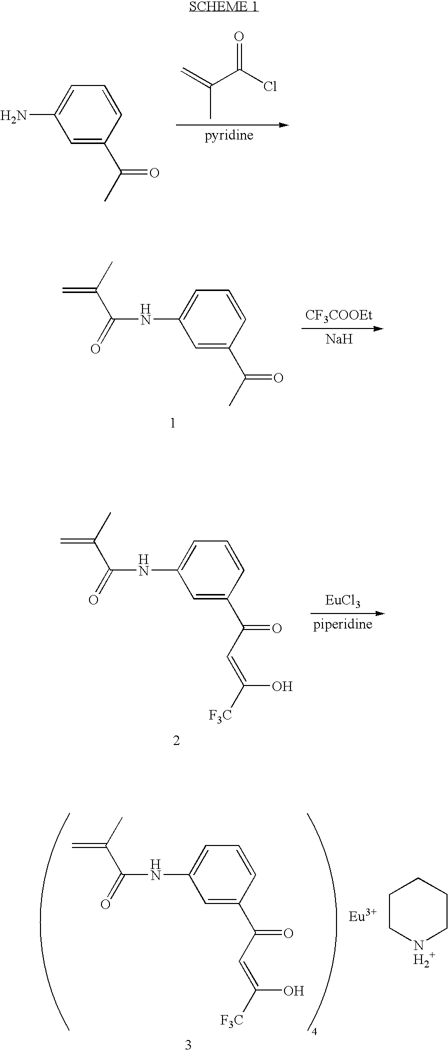

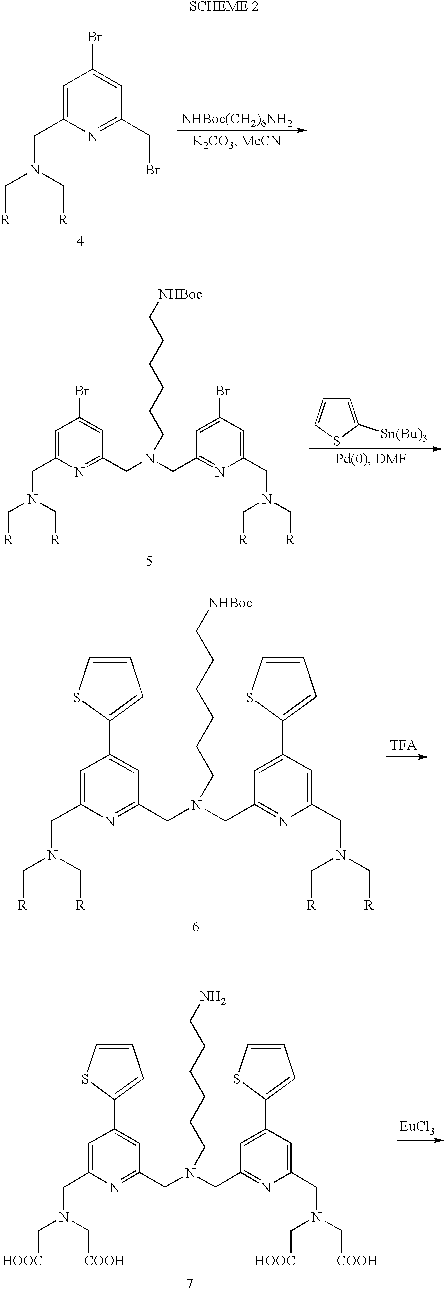

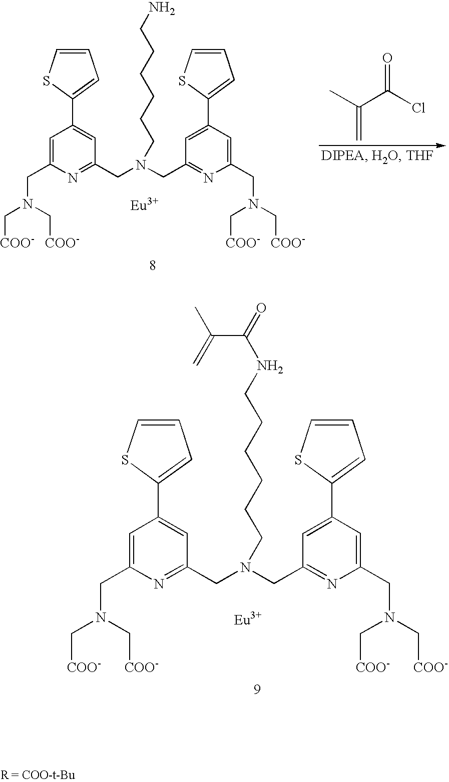

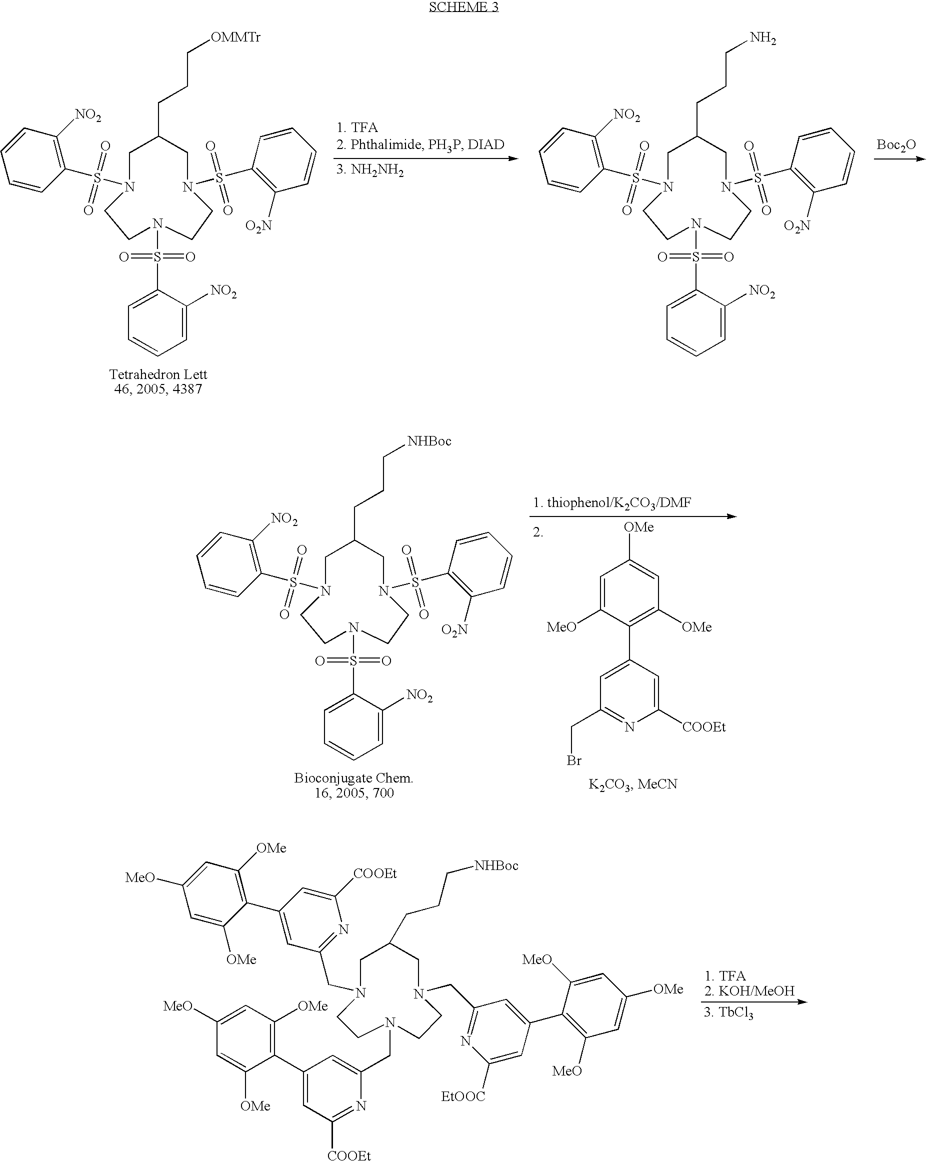

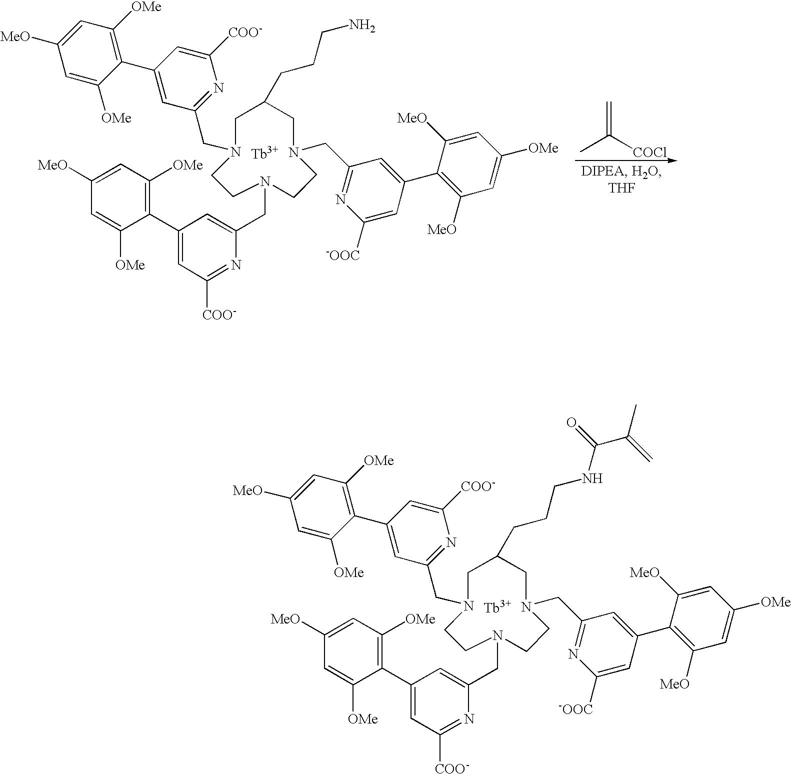

1. A method for preparing a particle comprising a lanthanide chelate, comprising polymerizing a lanthanide chelate derivative with one or more monomers, wherein the lanthanide chelate derivative has the formula M-L-Y (I), wherein Y is a lanthanide chelate; L is a linker and M is a polymerizable moiety.

2. The method according to claim 1 wherein the polymerizable moiety M is selected from pendent vinyl groups, acrylate groups, methacrylate groups, ethacrylate groups, 2-phenylacrylate groups, vinylketone groups, acrylamide groups, methacrylamide groups, itaconate groups and styrene groups.

3. The method according to claim 2 wherein M is methacrylamide or acrylamide.

4. The method according to claim 1 wherein the lanthanide chelate derivative is a compound of formula (I) and the one or more monomers are selected from styrene, vinyl alcohol, acrylic acid, metacrylic acid monomers or esters or amides thereof.

5. The method according to claim 1 wherein the lanthanide chelate derivative is a compound of formula (I) and the one or more monomers are styrene monomers.

6. The method according to claim 1 wherein the lanthanide chelate is a compound of formula (I) selected from the group of: 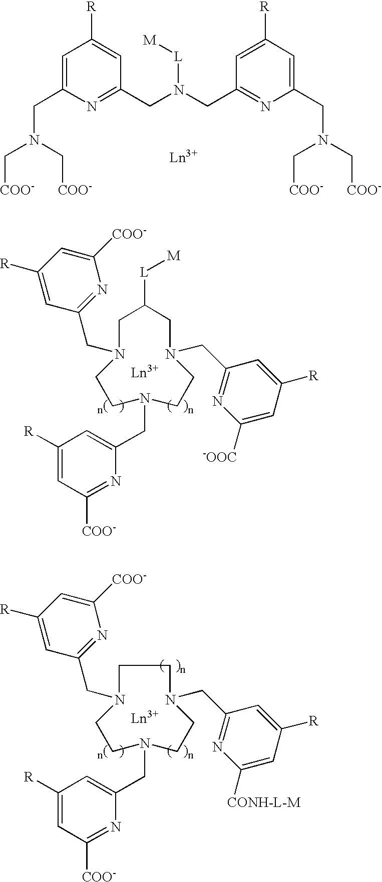
wherein
R is independently furyl, thiophenyl, or trialkoxyphenyl;
n is independently 1 or 2; and
L is a linker;
Ln is europium, terbium, samarium, dysprosium or gadolinium; and
M is a polymerizable moiety.

7. The method according to claim 1 wherein the linker -L- is formed from one to ten moieties, each moiety being selected from the group consisting of phenylene, alkyl containing 1-12 carbon atoms, ethynediyl(—C≡C—), ethylenediyl (—C═C—); ether (—O—), thioether (—S—), amide (—CO—NH— and —NH—CO— and —CO—NR′ and —NR′—CO—), carbonyl(—CO—), ester (—COO— and —OOC—), disulfide (—SS—), diaza, (—N═N—), thiourea (—NH—CS—NH—) or a tertiary amine (—NR′—), where R′ represents an alkyl containing less than 5 carbon atoms.

8. The method according to claim 1, wherein the particle has a diameter of less than 500 nm.

9. The method according to claim 1, wherein the polymerizing is performed in the presence of a cross-linking agent.

10. The method according to claim 9, wherein the cross-linking agent is divinylbenzene.

11. A compound of the formula M-L-Y (I), wherein Y is a lanthanide chelate; L is a linker and M is a polymerizable moiety.

12. The compound according to claim 11, wherein M is selected from pendent vinyl groups, acrylate groups, methacrylate groups, ethacrylate groups, 2-phenylacrylate groups, vinylketone groups, acrylamide groups, methacrylamide groups, itaconate groups and styrene groups.

13. The compound according to claim 12, wherein M is methacrylamide or acrylamide.

14. The compound according to claim 11, selected from the group of: 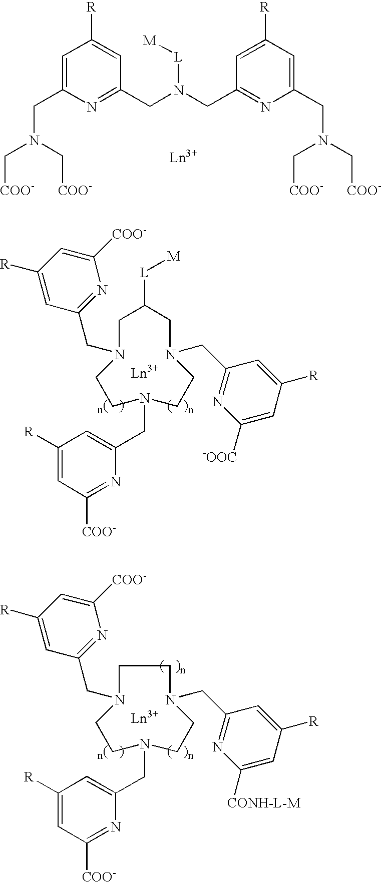
wherein
R is independently furyl, thiophenyl, or trialkoxyphenyl;
n is independently 1 or 2; and
L is a linker;
Ln is europium, terbium, samarium, dysprosium or gadolinium; and
M is a polymerizable moiety.

15. The compound according to claim 11 wherein the linker -L- is formed from one to ten moieties, each moiety being selected from the group consisting of phenylene, alkyl containing 1-12 carbon atoms, ethynediyl(—C≡C—), ethylenediyl(—C═C—); ether (—O—), thioether (—S—), amide (—CO—NH— and —NH—CO— and —CO—NR′ and —NR′—CO—), carbonyl(—CO—), ester (—COO— and —OOC—), disulfide (—SS—), diaza, (—N═N—), thiourea (—NH—CS—NH—) or a tertiary amine (—NR′—), where R′ represents an alkyl containing less than 5 carbon atoms.

16. A particle comprising a compound of formula (II)
Z-L-Y (II)
wherein,
Z is an organic polymer or copolymer
L is a linker; and
Y is a lanthanide chelate,
wherein the lanthanide chelate Y is covalently bound to Z via the linker L.

17. The particle according to claim 16 where the diameter of the particle is less than 500 nm.

18. The particle according to claim 16 wherein the linker -L- is formed from one to ten moieties, each moiety being selected from the group consisting of phenylene, alkyl containing 1-12 carbon atoms, ethynediyl(—C≡C—), ethylenediyl(—C═C—); ether (—O—), thioether (—S—), amide (—CO—NH— and —NH—CO— and —CO—NR′ and —NR′—CO—), carbonyl(—CO—), ester (—COO— and —OOC—), disulfide (—SS—), diaza, (—N═N—), thiourea (—NH—CS—NH—) or a tertiary amine (—NR′—), where R′ represents an alkyl containing less than 5 carbon atoms.

19. The particle according to claim 16 wherein L-Y of compound (II) is selected from 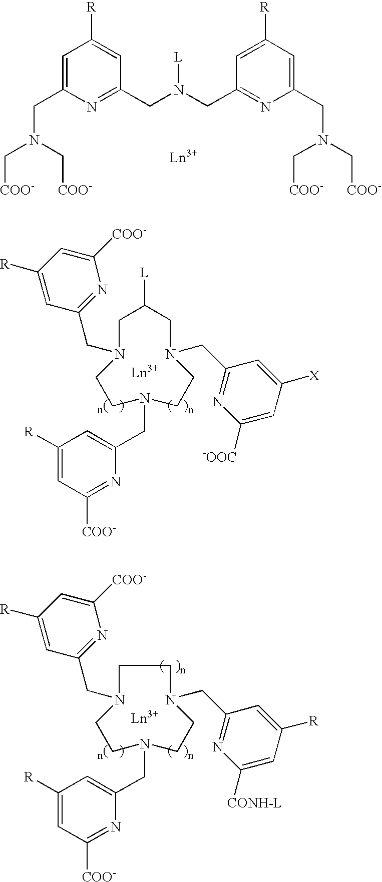
wherein
R is independently furyl, thiophenyl, or trialkoxyphenyl;
n is independently 1 or 2;
L is a linker;
and Ln is europium, terbium, samarium, dysprosium or gadolinium.

20. The particle according to claim 16 wherein the organic polymer or copolymer is constructed from monomers of vinyl, acrylate, methacrylate, ethacrylate, 2-phenylacrylate, vinylketone, vinyl alcohol, acrylamide, methacrylamide, itaconate or styrene.

21. The particle according to claim 16 constructed with a cross-linking agent.

22. The particle according to claim 21 wherein the cross-linking agent is divinylbenzene.
